(19) United States
(12) Patent Application Publication
(10) Pub. No.: US 2026/0311825 A1
(43) Pub. Date: Oct. 8, 2026

(54) SYSTEM AND METHOD OF GENERATING ICD CODES FROM UNSTRUCTURED DATA USING ARTIFICIAL INTELLIGENCE

(71) Applicant: Humana Inc., Louisville, KY (US)

(72) Inventors: Pei-Yau LUNG, Louisville, KY (US); Michael GAYHART, Louisville, KY (US); Molu SHI, Louisville, KY (US); Ravi SRIVASTAVA, Louisville, KY (US); Steven SCHWEINHART, Louisville, KY (US); Priscilla WU, Louisville, KY (US)

(21) Appl. No.: 19/098,290

(22) Filed: Apr. 2, 2025

(51) Int. Cl. G16H 10/60; G16H 70/20

(52) U.S. Cl. CPC G16H 10/60; G16H 70/20

(57) ABSTRACT

A method of preprocessing and applying a machine learning model to more efficiently assign disease classification codes to unstructured data. Patient records are broken down into smaller data units or sentences and the most relevant data units or sentences are filtered out using a unique combination of a bi-encoder and cross-encoder. The filtered data units or sentences are then inputted into an appropriately trained neural network to output at least one disease classification code for the patient records. Domain-specific knowledge is then applied to filter and correct model outputs to ensure high quality coding.

### BACKGROUND OF THE INVENTION

The present invention is directed to a new and novel system and method for using generative AI (artificial intelligence) to more efficiently assign ICD codes to unstructured data. ICD (International Classification of Diseases) codes are standardized alphanumeric codes that uniquely identify diseases, symptoms, abnormal findings, and procedures. They were developed by the World Health Organization and have evolved over time (with versions like ICD-10 and ICD-11) to capture a wide array of medical conditions accurately.

In the health insurance arena, ICD codes serve several important functions:
Billing and Reimbursement: Health care providers submit ICD codes to insurance companies to document diagnoses and justify the medical services rendered. These codes are essential for processing claims and ensuring that the services provided are appropriately reimbursed.
Statistical and Epidemiological Analysis: Insurers and public health agencies use ICD-coded data to track disease prevalence, evaluate treatment outcomes, and identify public health trends.
Regulatory and Administrative Purposes: The codes help maintain consistency and transparency in the documentation of medical conditions, facilitating audits, quality control, and compliance with health regulations.

Traditionally, ICD coding is performed manually by trained and often certified medical coders. The process involves:
Reviewing Patient Records: Coders carefully read through a patient's health records, which may include clinical notes, lab results, discharge summaries, and other documentation.
Interpreting Medical Terminology: They interpret the clinical language and identify key diagnoses, symptoms, and procedures that need to be coded.
Assigning the Correct Code: Based on detailed coding manuals and guidelines, the coder selects the most accurate ICD code(s) that correspond to the documented medical conditions.
Ensuring Compliance: The codes, in certain environments, must comply with both internal policies and external regulatory requirements, which adds an extra layer of complexity to the process.

The manual coding process is not only time-consuming but also costly and prone to errors:
Labor Intensive: With the increasing volume of patient data, especially from unstructured sources like free-text clinical notes, the manual process becomes a significant bottleneck.
Human Error: Even experienced coders can make mistakes, which may lead to incorrect billing, claim denials, or misrepresentation of a patient's condition.
Costly: Manual coding requires highly trained professionals, and the time involved directly translates to higher operational costs.
Scalability Issues: As the healthcare industry continues to generate vast amounts of data, the need for scalable, efficient, and automated coding solutions becomes paramount.

For example, in one manual process, the process proceeds as follows:
1. Care Provision:
   A clinician visits the patient at their home to provide care and then documents the visit and any relevant patient information.
2. Case Management (CM):
   The case manager reviews the clinician's documentation for accuracy and completeness, offering feedback and comments to ensure high-quality patient care.
3. Coding:
   Licensed coders examine the patient's medical records and assign appropriate codes for diagnoses and procedures, which are essential for billing and insurance purposes.
4. Clinician Approval:
   The clinician checks the coders' work to ensure that the codes accurately reflect the patient's condition and the care provided. They must approve the coding before it can proceed.
5. Submit for Billing
   Once the clinician has approved the coding, the final coding output is submitted for billing. This step ensures that all necessary checks and approvals have been completed before the billing process begins.
      This multi-step process is time consuming, inefficient and prone to human error.

Given these challenges, there is a need for integrating advanced technologies such as natural language processing (NLP) and machine learning to automate the extraction and assignment of ICD codes. Such automated systems can analyze unstructured documents, reduce the workload on human coders, improve accuracy, and streamline the billing process, ultimately leading to better resource allocation and enhanced patient care.

### SUMMARY

In one embodiment of the invention, the invention is a method of preprocessing and applying a machine learning model to more efficiently assign disease classification codes to unstructured data, comprising the steps of: preprocessing a set of patient records comprising the steps of: for each patient record in the set of patient records:
a. breaking down each patient record into smaller data units;
b. pairing each data unit with a prompt, wherein the prompt is question or other textual string designed to find specific information;
c. inputting the prompt and each data unit into a bi-encoder model to provide a first similarity score that indicates how similar each data unit is related to the prompt;
d. filtering out a first subset of data units based on the first similarity score;
e. inputting the prompt, or a similar or related prompt, with each of the data units in the first subset of data units into a cross-encoder model to provide a second similarity score;
f. filtering out a second subset of data units from the first subset of data units based on the second similarity score wherein the second subset of data units are considered the most relevant data units to the prompt;
   converting the second subset of data units into input data in a predetermined format, inputting the formatted input data into a predetermined pretrained neural network, and outputting at least one disease classification code related to the second subset of data units.

The features and advantages described in the specification are not all inclusive and in particular, many additional features and advantages of the present invention will be apparent from the following more detailed description of the particular embodiments, as illustrated in the accompanying drawings.

### BRIEF DESCRIPTION OF DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 illustrates one embodiment of the process flow of the present invention.

FIG. 2 illustrates one embodiment of a pre-processing workflow of the present invention for parsing out relevant information from large amounts of unstructured data.

FIG. 3 illustrates one embodiment of an ICD coding workflow of the present invention using novel generative AI techniques.

FIG. 4 illustrates one embodiment of a post-processing workflow of the present invention using domain-specific knowledge to filter and correct model outputs to ensure high quality coding.

FIG. 5 illustrates one post-processing example process flow applying the teachings of the present invention.

### DETAILED DESCRIPTION

The following detailed description of the example embodiments refers to the accompanying figures that form a part thereof. The detailed description provides explanations by way of exemplary embodiments. It is to be understood that other embodiments may be used having changes that incorporate the scope of the present invention without departing from the spirit of the invention. One skilled in the art may readily recognize from the following description that alternative embodiments of the structures and methods illustrated herein may be employed without departing from the principles described herein.

The present invention described below is one method of more efficiently assigning ICD codes to information or data in a document, including unstructured documents, using a particular process for pre-processing of the relevant data or documents using a semantic search method using pretrained models, generating ICD codes using generative artificial intelligence (AI) and post-processing using an ICD knowledge base and filtering as described more fully below.

The present system processes various clinical documents commonly used in healthcare, including, for example, referral documents, related attachments, and physician visit notes. These documents are preferably text-based and can be in formats such as the portable document format (PDFs) or scanned faxes, provided they can be converted to text via Optical Character Recognition (OCR). The preferred system is optimized for textual content as opposed to non-textual content like images, handwritten notes, or complex tables without prior text conversion.

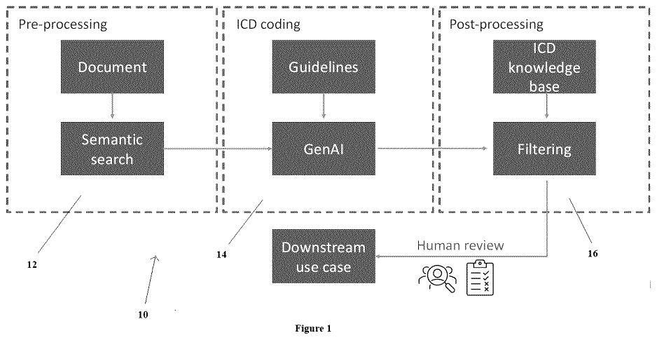

FIG. 1 illustrates one embodiment of the process flow 10 of the present invention. The process flow is preferably comprised of a pre-processing step 12, an ICD coding step 14 using generative AI, and a post-processing step 16 that uses domain-specific knowledge to filter and correct model outputs to ensure high quality coding.

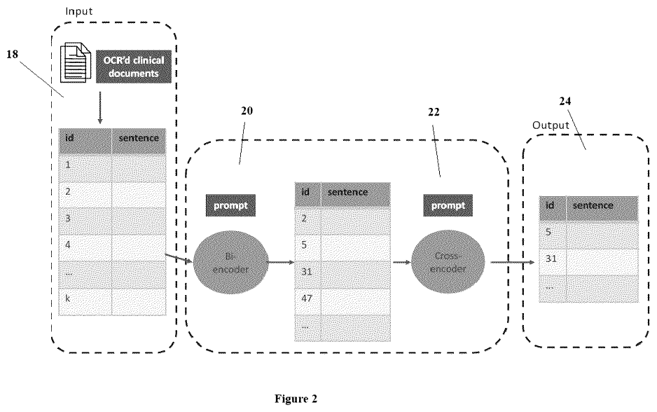

FIG. 2 illustrates one embodiment of a pre-processing workflow of the present invention for parsing out relevant information from large amounts of unstructured data. The pre-processing step is designed to identify and extract the most relevant parts of a clinical document for International Code of Diseases (ICD) coding. This is important because clinical documents often contain a lot of information, not all of which is pertinent to the coding process. By focusing on the most relevant parts, efficiency and accuracy of the ICD coding is greatly improved.

### Preprocessing

The preprocessing of the present invention is comprised of the following:
1. Breaking documents into sentences: Clinical documents (e.g., doctor's notes) are turned into smaller, manageable pieces, data units or text segments—e.g., sentences. This makes it easier to analyze the content step by step.
   By converting the documents into smaller, manageable pieces, the content can be analyzed more effectively. This granular approach allows the models to process each sentence independently, ensuring that the most relevant information is captured for accurate ICD coding.
2. Pairing each sentence with a prompt:
   A “prompt” is essentially a question or query designed to find specific information. For example, a prompt could be, “What is the patient's diagnosis?”
   Each sentence is compared to the prompt to see if it is relevant to or answers the query.

A prompt serves as a specific question or query aimed at extracting particular information from the clinical document. Each sentence from the document is compared to the prompt to determine if it provides a relevant answer (each sentence of each document is preferably compared with each prompt during the semantic search process).

In this context, “similar” preferably means that the sentence is related to or relevant to the prompt. The bi-encoder calculates the cosine similarity score to measure the degree of relevance between the sentence and the prompt. A high similarity score indicates that the sentence is likely to contain information that answers or is pertinent to the query posed by the prompt.

### Bi-Encoder and Cross-Encoder

The preprocessing step of the present invention also comprises the following steps of parsing the sentence and prompt through a first bi-encoder filter and then a subsequent cross-encoder filter.
Bi-encoder: This is a first filter. Each sentence is paired with a specific prompt and processed by a bi-encoder model that has been pretrained. The pretrained model of the present invention looks at a sentence and the prompt, converts them into numerical representations (called embeddings), and calculates a similarity score. If the sentence is somewhat related to the prompt, it moves to the next stage.
Cross-encoder: This is a more detailed filter. It takes the selected sentences (e.g., those filtered through from the bi-encoder step) and assigns them a finer relevance score, which helps ensure that only the most relevant sentences are chosen. Sentences that receive a higher relevance score from the cross-encoder are considered the most relevant and are preferably selected as the final output of the semantic search process.
   Encoding turns text into numbers that computers can understand and compare. By comparing the embeddings of a sentence and a prompt, the system can decide how closely they are related.

Thus, the bi-encoder serves as the initial filter by converting both the sentence and the prompt into embeddings and calculating a similarity score to determine if the sentence is related to the prompt. Sentences that pass bi-encoder are then processed by the cross-encoder, which provides a relevance score. This two-step process ensures that only the most relevant sentences are selected for further processing.

### Fine-Tuning With Domain Data

The cross-encoder is preferably fine-tuned with medical data (like a medical insurance company's dataset) to perform better on healthcare-related tasks. Fine-tuning the cross-encoder with medical and health related data enhances its performance on healthcare-related tasks as it understands more healthcare-specific language and context, leading to more accurate and relevant results in the ICD coding process.

In the preferred embodiment, the bi-encoder is a pre-trained model that has been trained on millions of scientific papers. The bi-encoder is a neural network model based on transformer architectures. The pre-training process involves training the model to predict masked words in sentences and to understand the context of words within a document. This makes it suitable for tasks involving semantic search. In one embodiment, the bi-encoder can be used as is, without additional fine-tuning.

The cross-encoder is preferably fine-tuned using inpatient data housed at a health insurance company for example. The cross-encoder is also based on transformer architectures. The fine-tuning process involves training the cross-encoder to jointly encode pairs of inputs, allowing it to capture more intricate relationships and dependencies between them. This makes the cross-encoder particularly effective for tasks that require a deeper understanding of the interaction between input pairs. Specifically, in one embodiment, pairs of questions are used from the Milliman Care Guidelines (MCG) and the corresponding evidence in referral documents supporting the question as data points. MCG guidelines are used by health insurance companies and providers to determine the medical necessity, appropriateness and length of stay for treatments and medical procedures. This fine-tuning process allows the cross-encoder to learn specialized knowledge relevant to the healthcare setting, enhancing its performance on clinical documents.

Cosine similarity is preferable and primary metric used to determine the similarity between vectors in both the bi-encoder and cross-encoder models. Based on empirical validation, the thresholds are preferably set at 0.05 for the bi-encoder and 0.01 for the cross-encoder, as one example. In this example embodiment, low threshold values are used (e.g., 0.05/0.01 for bi-encoder and cross-encoder respectively) to ensure that partially relevant or ambiguous sentences are included in the next step for coding. The combination of bi-encoder and cross-encoder ensures that only the relevant sentences are retained, while noise and irrelevant information are excluded.

FIG. 2 illustrates one embodiment flow of the pre-processing step of the present invention, including the steps of inputting each sentence into the pretrained model 18 (showing a unique identification (ID) assigned to each sentence, parsing information using the bi-encoder 20 (e.g., comparison with prompts) and cross-encoder 22, which then filters out the most relevant sentences for each prompt at the output 24.

### Examples of Prompts

As one example, the following “prompts” were used in one example embodiment:
For the bi-encoder: “diagnosis, disease, problem, symptom, history, order, code”.
For the cross-encoder: “What is the diagnosis, disease, problem, symptom, or history of the patient?”

These prompts are preferably generated and defined based on experimentation and evaluation of their performance on relevant datasets. The system can be configured to dynamically create prompts based on the document context or other parameters and/or the system can be configured to provide prompts that are tailored to work appropriately with the particular data.

The prompts for the bi-encoder and cross-encoder models can be customized to suit different medical specialties or coding tasks. Users can create and test various sets of prompts tailored to specific specialties or tasks, selecting the ones that yield the best performance. This iterative process ensures that the models are optimized for the unique requirements of each medical domain.

This process of using a two-layer filtering process ensures that the second filtering layer (cross-encoder), only receives information that has already been parsed through the bi-encoder filtering which reduces compute needs and increases efficiency.

### ICD Coding

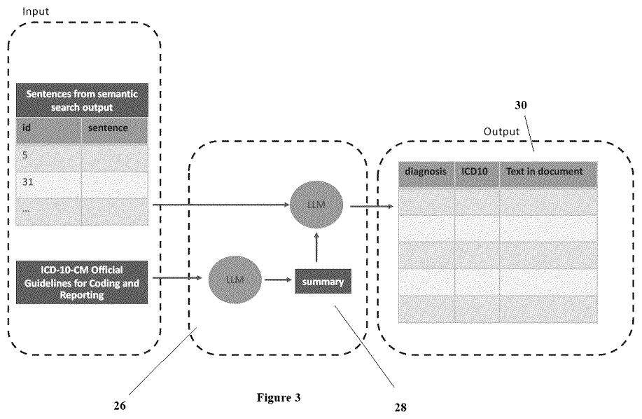

FIG. 3 illustrates one embodiment of an ICD coding workflow of the present invention using generative AI techniques. In this step of the present invention, the present invention is configured to take the information filtered out from the bi-encoder and cross-encoder and assign relevant ICD codes to the filtered information. In the preferred embodiment, the present invention breaks down the input data from the previous step into smaller, manageable chunks, ensuring that each chunk fed into the relevant model(s) does not exceed a specified maximum number of tokens. Tokens are the smallest units of text that a language model processes. They can range from a single character to an entire word, depending on the language model's tokenization process.

The input data, which are sentences from the semantic search output, is preferably broken down into smaller groupings of data or “chunks”. Each chunk is designed to not exceed a specified maximum number of tokens (500 in one example embodiment). The limit is preferably set to ensure efficient processing.

Smaller, more manageable chunks help the language model to focus on specific parts of the input data, reducing the risk of information overload and improving the model's ability to generate accurate and relevant diagnoses and ICD-10 codes. This “chunking” process also reduces latency, as the model can process smaller pieces of data more quickly and efficiently.

A diagnosis provides a detailed description of the patient's condition, while the ICD-10 code offers a standardized way to document and communicate that diagnosis across different healthcare systems.

### Example 1

Diagnosis: Type 2 Diabetes Mellitus with Diabetic Neuropathy
ICD-10: E11.40

### Example 2

Diagnosis: Chronic Obstructive Pulmonary Disease (COPD)
ICD-10: J44.9

In one embodiment, established large language models (LLMs), such as Chatgpt™ or Grok™, may be used. In the preferred embodiment, the “ICD-10-CM Official Guidelines for Coding and Reporting” are not used in the training process of the LLM. Instead, they are integrated into the inference process 26. These guidelines provide detailed descriptions for each ICD-10 code. Specifically, the guidelines are summarized and incorporated into the prompts used during inference 28. This includes key aspects such as reimbursement eligibility and symptomatic codes, ensuring that the LLM's outputs align with the official coding standards. In one embodiment, the output 30 is provided in tabular format including the fields of “diagnosis”, “ICD-10” code, and “Text in the document”.

Ambiguous or overlapping ICD codes are preferably handled in the post-processing step. The LLM in this step generates potential diagnoses and the corresponding ICD codes based on the clinical document. In the post-processing phase, the combination of bi-encoder and the knowledge base helps resolve ambiguities and ensures high-quality coding.

### Code Generation

In one embodiment, diagnosis and ICD codes are generated in JSON format. The structured format ensures that even if the output is truncated, the fields can still be parsed and understood correctly. For example, in one example system, the fields include:
diagnosis: patient positive diagnosis
code: ICD 10 code of positive diagnosis
date: date of service to record positive diagnosis
section: section head in the document recording positive diagnosis
citation: the exact words from the original document
type: primary or secondary of the positive diagnosis

It is possible that multiple codes are suggested to one diagnosis as there might be variations in the mentions of a diagnosis within the document. For example, the model identified “Acute myeloid leukemia in relapse” in the document and assigned ICD10 C92.00. It also identified a variant, “AML” and assigned the same diagnosis but different ICD10 C92.01. This is preferably handled in the post-processing stage, where the most accurate and relevant code is selected based on cosine similarity scores and domain-specific adjustments.

### Domain-Specific Knowledge

Knowledge from medical datasets (e.g., from a health insurance company) can be integrated into the large language model. When updates to guidelines, for example “ICD-10-CM Official Guidelines for Coding and Reporting v2.0” are released, a new summary highlighting the key aspects of the updated guidelines can be generated. This updated summary is then incorporated into the prompts used during inference, ensuring that the model remains current with the latest medical standards and practices.

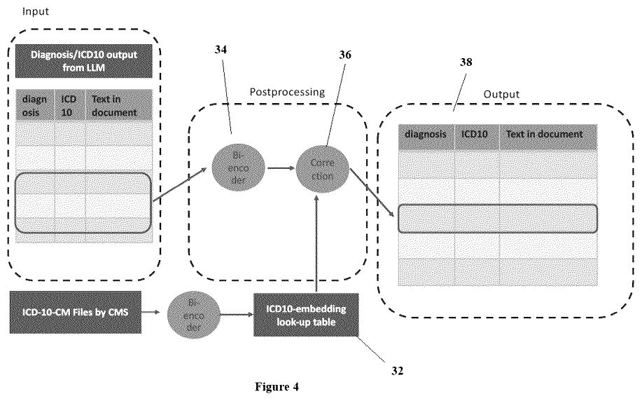

FIG. 4 illustrates one embodiment of a post-processing workflow of the present invention using domain-specific knowledge to filter and correct model outputs to ensure high quality coding.

### Post-Processing/Knowledge Base

The ICD knowledge base is preferably created and maintained by using the ICD-10-CM files provided by the Centers for Medicare & Medicaid Services (CMS) related to their guidelines as discussed above. These files contain detailed descriptions and codes for various diagnoses and procedures. By using a pretrained bi-encoder the descriptions are converted into embedding (numerical representations) that are stored in a lookup table for efficient comparisons 32. In the preferred embodiment, this bi-encoder is the same as the one used for preprocessing. To verify the model-generated ICD codes (generated from the ICD coding step described above), the diagnosis and the corresponding raw text (or sentence) from the original document are converted into embedding using the pretrained bi-encoder 34. These embeddings are then compared to the embeddings in the lookup table, preferably by calculating cosine similarity scores 36. The final ICD code is selected (outputted) based on the highest similarity score, ensuring that the chosen code most accurately matches the diagnosis and description in the original document 38. For ICD-10 codes not suggested by the LLM, in one embodiment, their similarity scores can be adjusted by multiplying them by a factor (a).

Regular updates from CMS are incorporated into the knowledge base using the same process. The following fields or attributes are preferably included in the knowledge base (e.g., descriptions, usage rules):
1. ICD10
2. Description
   The knowledge base is preferably updated whenever there are updates to the ICD-10-CM files from the CMS.

### Filtering and Validation

The comparison of embeddings of generated codes is preferably compared with the knowledge based using cosine similarity.

In the preferred embodiment, an adjustment factor is applied to cosine similarity scores. The adjustment factor is a multiplier (0<α<1) applied to the cosine similarity scores of ICD-10 codes that were not initially selected by the LLM. This factor reduces the weight of these codes to ensure a more accurate final selection. The value of the adjustment factor is determined through experiments to optimize performance for a specific use case (α=0.9 in one example case). Users can adjust this value based on their own requirements and experimental results.

In the preferred embodiment the present system is designed to produce relevant and accurate ICD codes. While it is feasible of detecting conflicts between suggested codes, in one embodiment, the final decision on resolving these conflicts is left to the coders. This approach ensures that the expertise and judgment of the coders are utilized to make the most appropriate final coding decisions.

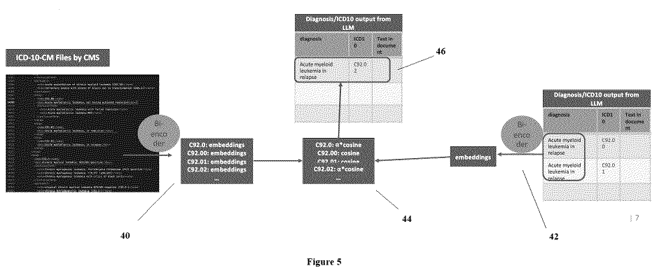

FIG. 5 illustrates one post-processing example process flow applying the teachings of the present invention. The example demonstrates how post-processing can correct an LLM-generated coding error. Initially, the LLM extracted “Acute myeloid leukemia in relapse” from a patient's referral document and coded it as both C92.00 and C92.01, even though the correct ICD-10 code is only C92.02 (and these two codes cannot be present together).

To fix this, the diagnosis “Acute myeloid leukemia in relapse” is converted into a numerical representation (embedding) using a bi-encoder. All relevant ICD-10 codes that begin with C92 are also converted into embeddings. The system calculates the cosine similarity between the diagnosis embedding and each code's embedding to find the best match.

An additional adjustment factor (α) is applied to similarity scores for all candidate codes except C92.00 and C92.01. After computing and adjusting these scores, the code with the highest similarity—C92.02 in this case—is chosen as the final, correct ICD-10 code. This process ensures that the output reflects the most accurate coding for the patient's diagnosis.

In summary, the following example illustrates how the system corrects errors, such as the wrong code for “acute myeloid leukemia in relapse”:
1. The LLM initially extracts the diagnosis “Acute myeloid leukemia in relapse” and incorrectly assigns the ICD-10 codes C92.00 and C92.01.
2. The diagnosis text “Acute myeloid leukemia in relapse” is sent to the bi-encoder to generate its embeddings 40.
3. Since LLM assigned C92.00 and C92.01, all ICD-10 codes starting with C92 are considered as potential candidates. The descriptions of each of these candidates are converted into embeddings using the bi-encoder respectively 42.
4. The embeddings from step 2 is compared with the embeddings of each candidate code from step 3 by calculating cosine similarity scores. All candidates except C92.00 and C92.01 are multiplied by a factor α (0.9 in this case) 44.
5. The ICD-10 code with the highest score is selected. In this example, C92.02 has the highest score and is selected as the final output 46 (for this particular example, even though an adjustment factor was applied to C92.02, as it was not one of the ICD codes selected by the initial ICD coding process, it had the highest similarity score and thus was selected as the final ICD code).

### Additional Considerations Embodiments

Certain portions of this description present embodiments using algorithms and symbolic representations of operations on information. Such representations are standard in the data processing arts and effectively convey the core substance of the invention to those skilled in the art. While described functionally, computationally, or logically, these operations are meant to be implemented by computer programs, electrical circuits, microcode, or similar means. For convenience, these arrangements of operations are sometimes referred to as modules, without any loss of generality. Accordingly, the described operations and associated modules may be embodied in software, firmware, hardware, or any combination thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Although the aforementioned describes embodiments of the invention, the invention is not so restricted. It will be apparent to those skilled in the art that various modifications and variations can be made to the disclosed embodiments of the present invention without departing from the scope or spirit of the invention. Accordingly, these other [medication modules, medical carts, height adjustment mechanisms, keyless entry systems, and methods of medication transport] are fully within the scope of the claimed invention. Therefore, it should be understood that the apparatuses and methods described herein are illustrative only and are not limiting upon the scope of the invention, which is indicated by the following claims.

1. A method of preprocessing and applying a machine learning model to more efficiently assign disease classification codes to unstructured data, comprising the steps of:
preprocessing a set of patient records comprising the steps of: for each patient record in the set of patient records:
a. breaking down each patient record into smaller data units;
b. pairing each data unit with a prompt, wherein the prompt is question or other textual string designed to find specific information;
c. inputting the prompt and each data unit into a bi-encoder model to provide a first similarity score that indicates how similar each data unit is related to the prompt;
d. filtering out a first subset of data units based on the first similarity score;
e. inputting the prompt, or a similar or related prompt, with each of the data units in the first subset of data units into a cross-encoder model to provide a second similarity score;
f. filtering out a second subset of data units from the first subset of data units based on the second similarity score wherein the second subset of data units are considered the most relevant data units to the prompt;
converting the second subset of data units into input data in a predetermined format;
inputting the formatted input data into a predetermined, pretrained neural network;
assigning and outputting at least one disease classification code related to the second subset of data units; and
generating at least one positive diagnosis related to the at least one disease classification code related to the second subset of data units and outputting the at least one positive diagnosis with the at least one disease classification code.

2. The method of claim 1, wherein the disease classification codes are International Code of Diseases (ICD) codes.

3. The method of claim 1, further comprising the step of:
fine-tuning the cross-encoder model with a predetermined medical dataset to provide for more accurate filtering.

4. The method of claim 1, further comprising the step of:
training the cross-encoder model by pairing specific prompts from predetermined specialized healthcare guidelines with each data unit from each patient record.

5. The method of claim 1, further comprising the steps of:
determining a first cosine similarity score to measure the degree of relevance between the prompt and each data unit inputted into the bi-encoder model;
filtering out a first subset of data units that have a first cosine similarity score above a predetermined first threshold value;
determining a second cosine similarity score to measure the degree of relevance between the prompt, or the similar or related prompt, and each of the data units in the first subset of data units inputted into the cross-encoder model; and
filtering out a second subset of data units that have a second cosine similarity score above a predetermined second threshold value.

6. The method of claim 1, further comprising the step of:
training the bi-encoder model with a dataset of scientific papers to predict a masked words in data unit of a first training dataset to understand the context of words within each patient record.

7. (canceled)

8. The method of claim 1, further comprising the step of:
incorporating a set of predetermined guidelines for assigning or coding disease classification codes with the input data into the predetermined pretrained neural network to more accurately predict the at least one disease classification code related to the second subset of data units.

9. The method of claim 1, further comprising the steps of:
outputting a second disease classification code associated with the at least one positive diagnosis;
embedding descriptions and codes for various diagnosis from a predetermined knowledge base;
embedding the at least one positive diagnosis;
comparing the embedded at least one positive diagnosis with the embedded descriptions and codes;
determining a set of similarity scores based on comparisons between the at least one positive diagnosis with the embedded descriptions and codes;
applying an adjustment factor to all of the similarity scores in the set of similarity scores except those associated with the at least one disease classification code and the second disease classification code; and
selecting a final disease classification code that is associated with the highest similarity score.

10. The method of claim 9, wherein the adjustment factor is applied to reduce weighting of non-selected codes or those that were not outputted with the at least one positive diagnosis to ensure a more accurate selection.

11. A method of preprocessing and applying a machine learning model to more efficiently assign disease classification codes to unstructured data, comprising the steps of:
preprocessing a set of patient records comprising the steps of: for each patient record in the set of patient records:
a. breaking down each patient record into smaller data units;
b. pairing each data unit with a prompt, wherein the prompt is question or other textual string designed to find specific information;
c. inputting the prompt and each data unit into a bi-encoder model to provide a first similarity score that indicates how similar each data unit is related to the prompt;
d. filtering out a first subset of data units that based on the first similarity score;
e. inputting the prompt, or a similar or related prompt, with each of the data units in the first subset of data units into a cross-encoder model to provide a second similarity score;
f. filtering out a second subset of data units from the first subset of data units based on the second similarity score wherein the second subset of data units are considered the most relevant data units to the prompt;
converting the second subset of data units into input data in a predetermined format;
inputting the formatted input data into a predetermined, pretrained neural network;
assigning and outputting at least one disease classification code related to the second subset of data units;
generating at least one positive diagnosis related to the at least one disease classification code related to the second subset of data units and outputting the at least one positive diagnosis with the at least one disease classification code;
outputting a second disease classification code associated with the at least one positive diagnosis;
embedding descriptions and codes for various diagnosis from a predetermined knowledge base;
embedding the at least one positive diagnosis;
comparing the embedded at least one positive diagnosis with the embedded descriptions and codes;
determining a set of similarity scores based on comparisons between the at least one positive diagnosis with the embedded descriptions and codes;
applying an adjustment factor to all of the similarity scores in the set of similarity scores except those associated with the at least one disease classification code and the second disease classification code; and
selecting and outputting a final disease classification code that is associated with the highest similarity score.

12. The method of claim 11, further comprising the step of:
incorporating a set of predetermined guidelines for assigning or coding disease classification codes with the input data into the predetermined pretrained neural network to more accurately predict the at least one disease classification code related to the second subset of data units.

13. The method of claim 11, wherein the adjustment factor is applied to reduce weighting of non-selected codes or those that were not outputted with the at least one positive diagnosis to ensure a more accurate selection.

14. The method of claim 11, further comprising the step of:
fine-tuning the cross-encoder model with a predetermined medical dataset to provide for more accurate filtering.

15. The method of claim 11, further comprising the step of:
training the cross-encoder model by pairing specific prompts from predetermined specialized healthcare guidelines with each data unit from each patient record.

16. The method of claim 11, further comprising the steps of:
determining a first cosine similarity score to measure the degree of relevance between the prompt and each data unit inputted into the bi-encoder model;
filtering out a first subset of data units that have a first cosine similarity score above a predetermined first threshold value;
determining a second cosine similarity score to measure the degree of relevance between the prompt, or the similar or related prompt, and each of the data units in the first subset of data units inputted into the cross-encoder model; and
filtering out a second subset of data units that have a second cosine similarity score above a predetermined second threshold value.

17. The method of claim 11, further comprising the step of:
training the bi-encoder model with a dataset of scientific papers to predict a masked words in data unit of a first training dataset to understand the context of words within each patient record.

18. A method of preprocessing and applying a machine learning model to more efficiently assign disease classification codes to unstructured data, comprising the steps of:
preprocessing a set of patient records comprising the steps of: for each patient record in the set of patient records:
a. breaking down each patient record into smaller data units;
b. pairing each data unit with a prompt, wherein the prompt is question or other textual string designed to find specific information;
c. inputting the prompt and each data unit into an encoder model to provide a similarity score that indicates how similar each data unit is related to the prompt;
d. filtering out a subset of data units based on the similarity score;
converting the subset of data units into input data in a predetermined format;
inputting the formatted input data into a predetermined, pretrained neural network;
assigning and outputting at least one disease classification code related to the second subset of data units;
generating at least one positive diagnosis related to the at least one disease classification code related to the second subset of data units and outputting the at least one positive diagnosis with the at least one disease classification code;
outputting a second disease classification code associated with the at least one positive diagnosis;
embedding descriptions and codes for various diagnosis from a predetermined knowledge base;
embedding the at least one positive diagnosis;
comparing the embedded at least one positive diagnosis with the embedded descriptions and codes;
determining a set of similarity scores based on comparisons between the at least one positive diagnosis with the embedded descriptions and codes;
applying an adjustment factor to all of the similarity scores in the set of similarity scores except those associated with the at least one disease classification code and the second disease classification code; and
selecting and outputting a final disease classification code that is associated with the highest similarity score.

19. The method of claim 18, further comprising the step of:
incorporating a set of predetermined guidelines for assigning or coding disease classification codes with the input data into the predetermined pretrained neural network to more accurately predict the at least one disease classification code related to the second subset of data units.

20. The method of claim 18, wherein the adjustment factor is applied to reduce weighting of non-selected codes or those that were not outputted with the at least one positive diagnosis to ensure a more accurate selection.
